(19) United States
(12) Patent Application Publication
(10) Pub. No.: US 2025/0217982 A1
(43) Pub. Date: Jul. 3, 2025

(54) Systems And Methods For Quantifying Blood Loss With A Surgical Sponge Management System

(71) Applicant: Stryker Corporation, Portage, MI (US)

(72) Inventors: Brian James VanDerWoude, Portage, MI (US); Mayank Kumar, San Jose, CA (US); Keng-Tsai Lin, San Jose, CA (US); Kevin J. Miller, Mountain View, CA (US); Nikhil Sharma, Redwood City, CA (US); Shubham Patil, Palo Alto, CA (US); Siddarth Satish, Portola Valley, CA (US)

(73) Assignee: Stryker Corporation, Portage, MI (US)

(21) Appl. No.: 18/847,866

(22) PCT Filed: Mar. 17, 2023
(86) PCT No.: PCT/US2023/015509

§ 371 Date: Sep. 17, 2024

Related U.S. Application Data
(60) Provisional application No. 63/321,400, filed on Mar. 18, 2022.

(51) Int. Cl. G06T 7/00; A61B 5/00; A61B 5/02; A61F 13/36; A61F 13/42; G06K 7/10; G06T 7/11; G06T 7/62; G16H 40/20

(52) U.S. Cl. CPC G06T 7/0014; A61B 5/02042; A61B 5/6847; A61F 13/36; A61F 13/42; G06T 7/11; G06T 7/62; G16H 40/20; A61F 2013/424; G06K 7/10297; G06T 2200/24; G06T 2207/20084; G06T 2207/30168; G06T 2207/30196

(57) ABSTRACT

Systems and methods for quantifying blood loss with a surgical sponge management system. A data reader detects a tag of the surgical sponge, and one or more processors identify a sponge type of the surgical sponge based on a unique identifier. A presentation window is displayed on a user interface with dimensions of the presentation window based on the identified sponge type. The processor(s) determine whether characteristics of the surgical sponge satisfy acceptance criteria based on the identified type of the surgical sponge. The acceptance criteria may include sponge presence, folded sponge, partial sponge, presentation distance, correct sponge, moving sponge, mask validation, and sponge supervision. An optical sensor captures a color image of the surgical sponge once the characteristics satisfy the acceptance criteria. A volume of the blood or blood component on the surgical sponge is estimated based on the color image, and displayed on a user interface.

### PRIORITY CLAIM

This application claims priority to and all the benefits of U.S. Provisional Patent Application No. 63/321,400, filed Mar. 18, 2022, the entire contents of which are hereby incorporated by reference.

### BACKGROUND

Managing surgical sponges is an area of importance in the modern surgical suite, most importantly, ensuring that no surgical sponges (or other objects) are inadvertently retained inside a patient or otherwise misplaced. In the past, healthcare professionals have relied upon manual sorting and counting of surgical sponges. More recently, surgical sponge management systems have utilized electronics to assist with counting the surgical sponges. One such system is sold under the tradenames SurgiCount and SurgiCount+ by Stryker Corporation (Kalamazoo, Mich.) and disclosed in commonly-owned United States Patent Publication No. 2013/0088354, published Apr. 11, 2013, hereby incorporated by reference in its entirety, in which a radiofrequency identification (RFID) reader detects RFID tags on the surgical sponges.

Determining blood loss during surgery may be used to monitor intraoperative patient health. It is known to estimate blood loss during surgery by visual evaluation of the surgical sponges and other fluid-absorbing articles (e.g., surgical gowns, bedding, or drapes), which is inherently subjective and therefore prone to human error. It is also known to estimate blood loss during surgery by weighing the surgical sponges on a scale in bulk, which requires the user transport the surgical sponges to the scale to be weighed. Advances in imaging and computing have provided for quantifying blood loss by capturing an image of the surgical sponge. One such system is sold under the tradename Triton by Gauss Surgical, Inc. (Menlo Park, Calif.) and disclosed in commonly-owned U.S. Pat. No. 8,897,523, issued Nov. 25, 2014, and U.S. Pat. No. 10,424,060, issued Sep. 24, 2019, the entire contents of each being hereby incorporated by reference.

The systems using image-based blood quantification may not adequately compensate for differing types of surgical sponges, and may not realize the advanced functionality of the surgical sponge management system. Therefore, there is a need in the art to provide for an improved system and methods that overcome the aforementioned disadvantages and does so in an intuitive and seamless workflow to the user who may be accustomed to existing surgical protocols.

### SUMMARY

The present disclosure is generally directed to analysis of surgical articles through image-based processing that is based on an article type of the surgical article being known. The primary embodiment is described in the context of a surgical sponge management system and methods for quantifying blood loss on surgical sponges bloodied during a surgical procedure. However, it is contemplated that the objects of the present disclosure may be applied for surgical articles other than surgical sponges, including sharps, tools (powered or manual), and other instruments. Among other advantages, the functionality of the system and methods obviate the need for the user to determine, recall, and/or manually enter an article type, and/or engage any screen or device to trigger capture of an image for analysis. For surgical sponges, the system is able to perform QBL on surgical sponges of differing sizes and plys, and may do so automatically and in real-time without requiring the user to meaningfully alter the workflow of counting out the surgical sponges. The need to transport the surgical sponges to a separate scale for weighing is likewise obviated. Further, the surgical sponge management system provides for QBL without any increase in footprint within the surgical suite, and accommodates the sponge sizes being imaged or scanned at a generally common distance from the camera and at a convenient height for the user. For other surgical articles, the system is able to confirm the article being presented to the system is of a same article type as previously identified. The image processing may further facilitate identification of damaged articles, used versus unused article status, article cleaning status, automated article counting, and the like. Therefore, the terminology associated with the sponge-based methods described herein may be modified to apply to non-sponge-based surgical articles as well.

The surgical sponge management system may include a user interface, a data reader, one or more processors, memory, communications device, and/or other hardware. The user interface may be a tablet with a touchscreen display. A camera including an optical sensor and optionally a depth sensor may be integrated on the tablet. Neural networks may be implemented or executed on the processor of the tablet, on one or more processors of hardware coupled to or remote from the tablet, via cloud computing, combinations thereof, or the like.

The data reader may be used either as a handheld device, or when supported by a cradle. The data reader is configured to detect tags associated with the surgical sponges. The data reader may a radiofrequency (RFID) reader configured to detect RFID tags. The surgical sponges may be counted in by causing the tag to be detected by the data reader. The detected tag transmits identifying data to the processor. The tag includes at least one unique identifier indicative of characteristics of the surgical sponge. The unique identifier may include sponge size or type. The processor is configured to determine a type of the surgical sponge based on the unique identifier. The processor may index a counter to reflect the one or more surgical sponges being counted in to surgical procedure. The counter may be displayed on the user interface.

The method includes counting out the surgical sponge. The tag of the surgical sponge is detected by the data reader, and the unique identifier stored on the tag are received by the processor. The processor identifies the sponge type of the surgical sponge from a database of predetermined sponge types based on the unique identifier. The user interface may index and display the counter including an updated count for each sponge type that has been counted in to and counted out of the surgical procedure.

The method may include triggering the system for sponge imaging. The camera may be automatically activated in response to the detection of the tag with the data reader. An image feed, as detected by the camera may be displayed on the user interface. The image feed may be based on data being received from the optical sensor and/or the depth sensor. The image feed may include image frames forming a video of the user holding the sponge in a field of view of the optical sensor. The image feed may include graphical augmentations or indicia based on data from the optical sensor and/or the depth sensor. The displaying of the image feed may be automatically performed in response to the detection of the tag with the data reader.

The method may include displaying, on the user interface, the presentation window overlying the image feed being captured by the camera. The dimensions of the presentation window are based on the identified sponge type of the surgical sponge corresponding to the detected tag of the surgical sponge being counted out. The processor may determine acceptance criteria based on the type of surgical sponge—and the presentation window may be sized and scaled so that at least some of the acceptance criteria are met if the user presents the surgical sponge in a manner to at least substantially match or conform to the presentation window. Alternatively, the presentation window may not be displayed, but rather the processor may determine a dynamic presentation window based on an actual location of the sponge within the optical and/or depth sensors.

The image feed is processed or analyzed for satisfaction of the acceptance criteria or guardrails. The acceptance criteria are generally designed to ensure the QBL image of the surgical sponge captured by the optical sensor is high quality. The step includes analyzing each image frame (or every few image frames) of the image feed to ensure a stretched, correct surgical sponge is presented at the appropriate distance, unfolded, and not moving, among other acceptance criteria. If any one or more of the acceptance criteria remains unsatisfied, the processor prevents the camera from capturing the QBL image of the surgical sponge for QBL analysis. Once the acceptance criteria are determined to be satisfied, the method includes the step of acquiring, with the camera, the QBL image(s) of the surgical sponge. The QBL image may be the subsequent frame(s) of the image feed immediately following satisfaction of the acceptance criteria and output from a sponge recognition algorithm executing a sponge recognition state machine (SRSM). The QBL image is analyzed with a hemoglobin estimation engine for determination of a blood component (e.g., hemoglobin) of the liquid on the surgical sponge. The processor may extract a color component value (e.g., a redness value) from the QBL image, and execute a trained hemoglobin estimation algorithm implementing a hemoglobin estimation engine to determine the mass of the blood component on a pixel-by-pixel or other suitable basis. The hemoglobin estimation algorithm may be unique to the sponge type having been counted out. The hemoglobin estimation algorithm may be trained on extensive datasets for each sponge type to be compatible with the system. The blood loss may be determined based on the blood component. By using the sponge type to determine characteristics of the presentation window and the acceptance criteria, the image-based QBL may be applied to more or most types of surgical textiles, including those of differing sizes, plys, and/or colors.

For certain sponge types of lower plys, the sponge may need to be folded before imaging. The presentation window being displayed on the user interface may be used to guide the user to fold the surgical sponge. The method may also include the optional step of displaying, on the user interface, a folding protocol to the user. The presentation window may be dimensioned to approximate the dimensions of the surgical sponge when folded according to the folding protocol. The folding protocol may include guidance to facilitate the user folding the surgical sponge in the desired manner. The guidance may be textual instructions or graphical indicia, and/or audible content.

A sponge recognition algorithm is executed by the one or more processors and configured to process the images of the surgical sponges to detect the sponge and apply the acceptance criteria or guardrails. The sponge recognition algorithm may include image pre-processing, neural networks, labeling logic, guardrail algorithms, the SRSM, and any post-image processing. There may be at least two neural networks: a localization neural network and a segmentation neural network. The localization neural network is configured to, among other actions, detect whether or not there is a sponge in the image, and provide a bounding box around the detected sponge. The bounding box may include information about the detected sponge, for example, a width, height, x-shift and y-shift. Additionally, the localization neural network may be configured to determine if a non-stretched, or a partial sponge or no sponge is presented within the presentation window. The segmentation neural network provides per-pixel prediction of a sponge segmentation mask of whether a pixel is a sponge pixel or background pixel. The segmentation neural network may utilize data from both the optical sensor and the depth sensor, or alternative from only the optical sensor. A localization label receives output from the localization neural network, and a segmentation labeler receives output from the segmentation neural network. The labels are fed to the SRSM to identify video-based states based on dominant class predictions over a preset number of previous frames. The output of the sponge recognition algorithm is provided to the hemoglobin estimation algorithm.

First, the sponge recognition algorithm determines whether a surgical sponge is present. Next, the method includes determining whether the surgical sponge is folded by determining whether the surgical sponge is at least substantially square or rectangular. The localization neural network generates the bounding box of the sponge being detected. Based on outer edges of the bounding box, the localization labeler determines whether the surgical sponge is sufficiently square or rectangular. The determination may be associated with the sponge type. The step may include determining whether an aspect ratio of the presented surgical sponge is within an acceptable range based on the sponge type being counted out. The localization neural network provides the bounding box and compares a determined aspect ratio of the bounding box against the acceptable range of the aspect ratio for that sponge type. If the determined aspect ratio of the bounding box is outside of the acceptable range, the localization labeler determines the sponge is a folded sponge, and generates a localization label accordingly.

The method may include determining whether the sponge being presented is a partial sponge. If the bounding box of the sponge extends beyond the field of view of the camera, the localization labeler determines a partial sponge condition. Further, the localization labeler may determine whether the bounding box is too close to the presentation window. A margin, i.e., a pixel-based distance between the bounding box and the presentation window, may be determined, and compared against margin threshold values. If the determined margin is less than the margin threshold value, the localization labeler generates the localization label the presented surgical sponge is a partial sponge.

The sponge recognition algorithm may include determining whether the sponge is positioned too close or too far from the camera by determining whether the sponge is too large or too small, respectively. The localization labeler determines a pixel-based area of the bounding box, and compares the bounding box area against an acceptable range of bounding box area that is unique to each sponge type. The acceptable range of the bounding box area may approximate the presentation window. If the processor determines the bounding box area is greater than or less than the acceptable range of the bounding box area, the localization labeler generates a localization label that the sponge is too close or too far from the camera, respectively.

The segmentation neutral network outputs the segmentation mask in which sponge pixels and the background pixels are separated. The segmentation neutral network may run on the image frames of the image feed only for which the localization neural network has determined a sponge is present. The segmentation neural network may run in parallel or sequentially after the localization network to optimize processing speed and resource tradeoff. The method includes validating the sponge segmentation mask by determining a number of pixels in the sponge segmentation mask, and comparing the determined number of pixels against an acceptable range of pixels. The acceptable range of pixels is based on the sponge type, which again is identified by the processor with the counting out of with the data reader. If the segmentation mask, as determined by the segmentation neutral network, does not have a sufficient number of pixels, the segmentation mask is determined to be invalid. The segmentation labeler may generate a segmentation label accordingly.

A segmentation mask refinement algorithm may reject pixels from the segmentation mask that have different depth values that exceed a threshold when compared to most of the mask pixels. Differences in the depth values may be estimated as the distance between respective points and plane fitted to the depth map. The threshold may be the same or different for different sponge types. The depth data may be used to establish thresholds on acceptable presentation distance. The method may include determining whether the presented surgical sponge is too close or too far. The segmentation labeler may output a corresponding segmentation label that the presented surgical sponge too close or sponge too far if the average depth of all pixels belonging to the sponge segmentation mask is less than or greater than the minimum threshold or maximum threshold, respectively.

The segmentation labeler may utilize a depth map of depth data obtained from the depth sensor to determine real-world dimensions of the presented surgical sponge for assessing further acceptance criteria. The method may further include determining whether the presented surgical sponge is the correct sponge. By leveraging the depth map to determine the sponge area of the segmentation mask, the segmentation labeler compares the determined real-world sponge area against an acceptable range of sponge area, which is based on the sponge type being counted out. If the determined real-world sponge area of the segmentation mask is outside the acceptable range of the sponge area, the segmentation labeler outputs a wrong sponge label. The step may further include comparing a sponge compactness against a predetermined compactness threshold. The predetermined compactness threshold may be a fraction of the area of the bounding box that must be exceeded by the determined sponge area of the segmentation mask to be determined as correct.

The method may further include determining whether the presented surgical sponge is moving. A centroid of the sponge segmentation mask may be determined, and an average motion magnitude of the centroid may be determined within a predetermined number of image frames of the image feed. The step may include comparing the average motion magnitude against a centroid change threshold, which may be based on the sponge type being counted out. If the average motion magnitude is greater than the centroid change threshold, the segmentation labeler determines the presented surgical sponge to be moving too quickly, and the acceptance criteria to be not satisfied.

The segmentation and localization neural networks can be combined into a single neural network that outputs both the classification labels (e.g., no sponge, partial sponge, folded sponge, and full sponge) as well as segmentation masks and the associated bounding box around the detected sponge. Further, the segmentation and localization labeler can be combined to check for each of the guardrail as a sequence of condition check based on detected sponge feature and expected sponge type. The segmentation and localization neural networks may be executed concurrently or sequentially.

The method may include performing sponge supervision in which the user's hand, torso, neck, and other associated body parts is detected using a pose estimation neural network, as well as the location of the presented surgical sponge using the localizer neural network or the segmentation neural network. Once the person and the presented surgical sponge are detected and located, the pose estimation neural network is configured to determine whether the presented surgical sponge is being held properly in front of the torso. The sponge supervision algorithm may identify a body centerline, and a sponge vertical centerline, and further determine a horizontal shift in location of the presented surgical sponge relative to body centerline. One or more of the additional centerlines may be compared against a horizontal sponge centerline of the presented surgical sponge to determine a vertical shift of the presented surgical sponge. The horizontal shift and/or a vertical shift may be compared against a respective shift threshold relative and/or a predetermined, preferred position. Guidance may be provided on the user interface to instruct the user to present the surgical sponge in front of their torso if the shift(s) exceed the shift threshold. The depth map from the depth sensor may be used to distinguish between people in background and the foreground, and the pose estimation algorithm determines which of multiple people in the field of view of the camera is presenting the sponge.

The sponge recognition algorithm includes the sponge recognition state machine (SRSM). The SRSM is configured to receiving the labels from each of the localization labeler and the segmentation labeler and performs further processing prior to the camera capturing the QBL image. The SRSM algorithm determines whether the acceptance criteria or guardrails, executed potentially separately by the localization labeler and the sponge segmentation labeler, have been satisfied, by combining the aggregate state from both the localization and segmentation labeler. The SRSM algorithm may execute a smoothing procedure to aggregate the localization and segmentation labels using a sliding window with an image frame size. A most frequent aggregated sponge label is called the dominant label. The SRSM may indicate the QBL image is ready for capture and analysis by the hemoglobin estimation engine if the dominant label in the required consecutive frames satisfy the acceptance criteria. If no dominant label is identified within the last consecutive image frames, then the SRSM may generate an uncertain condition state.

Once the QBL image is captured, the SRSM algorithm may not permit further capturing of QBL images regardless of the immediate and dominant labels, at least until the dominant label identifies the sponge as no longer being or not present for a required number of consecutive image frames of the image feed. Once the sponge as no longer present for the required number of consecutive frames, the SRSM algorithm is configured to return to a state in which the localization neural network and segmentation neural network is readied to perform the methods disclosed herein.

### BRIEF DESCRIPTION OF THE DRAWINGS

FIG. 1 shows a surgical sponge management system. A user interface may be supported on a stand, and the system includes an optical sensor to capture a color image of a surgical sponge, and optionally a depth sensor.

FIG. 2 is a flowchart for quantifying blood loss according to an exemplary method of the present disclosure.

FIG. 3 is a perspective view of the user interface at a step of the method.

FIG. 4 is a perspective view of the user interface at another step of the method.

FIG. 5 is a perspective view of the user interface at another step of the method.

FIG. 6 is a flowchart including acceptance criteria according to a step of the method.

FIG. 7 is a schematic representation of neural networks and algorithms being implemented by one or more processors.

FIG. 8A is a flowchart for processing image frames of an image feed with a localization neural network according to an exemplary method of the present disclosure.

FIG. 8B is a flowchart for processing the image frames of the image feed with a segmentation neural network according to an exemplary method of the present disclosure.

FIG. 9 is an illustration for explanatory purposes of acceptance criteria.

FIG. 10 is an illustration for explanatory purposes of acceptance criteria.

FIG. 11 is an illustration for explanatory purposes of acceptance criteria.

FIG. 12 is an illustration for explanatory purposes of acceptance criteria.

FIG. 13 is an illustration for explanatory purposes of acceptance criteria.

### DETAILED DESCRIPTION

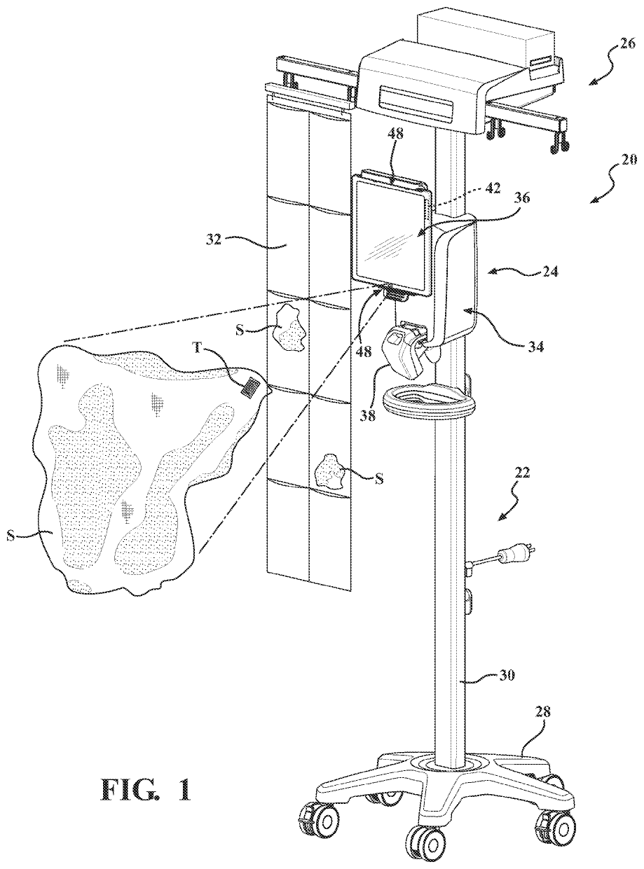

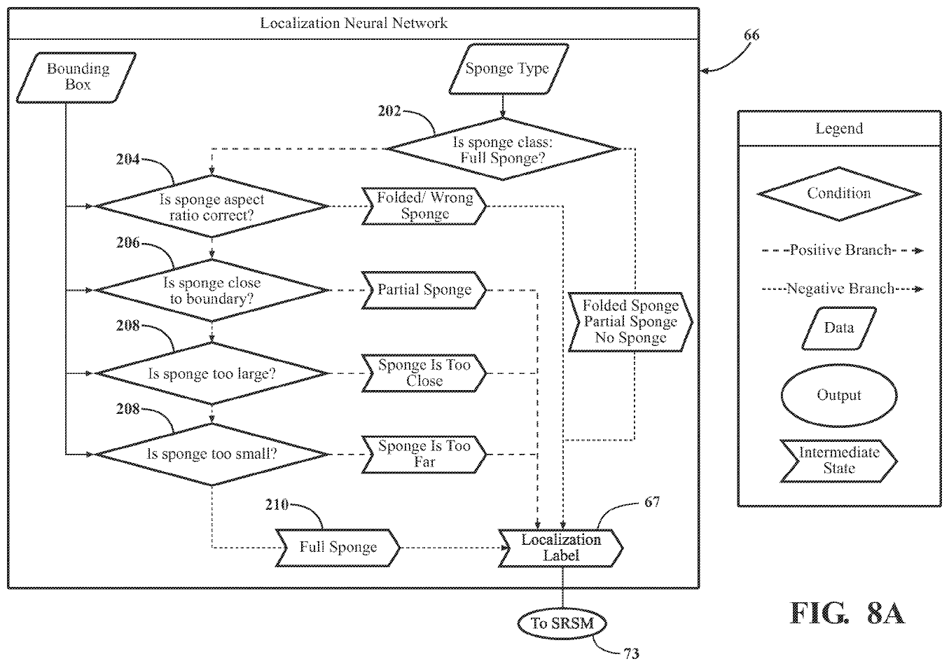

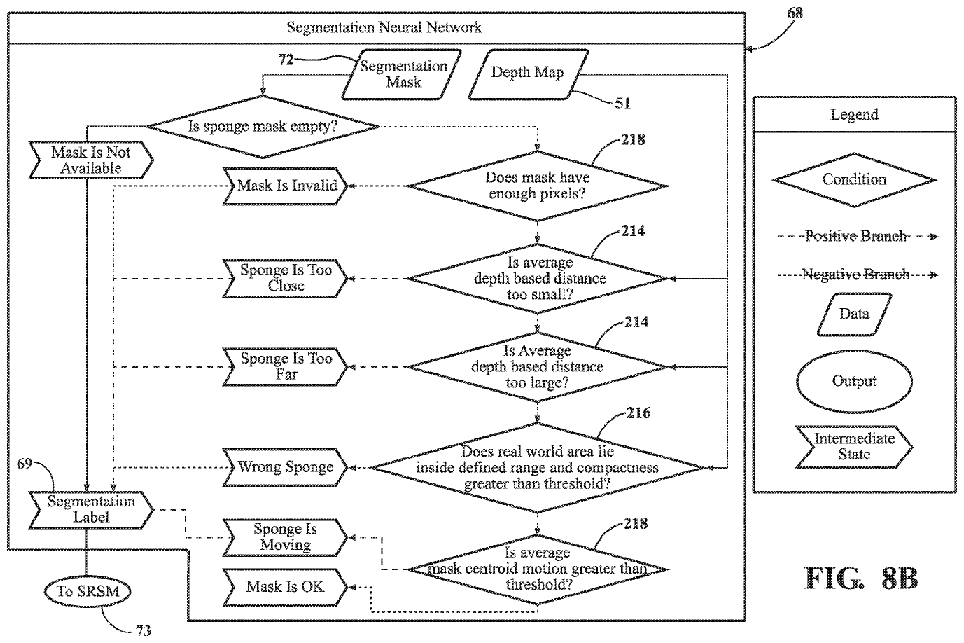

The present disclosure relates to a system for managing surgical sponges during a surgical procedure, and quantifying blood loss (QBL) associated with blood absorbed on the surgical sponges. Referring to FIG. 1, a surgical sponge management system 20 may include a stand 22, an electronics subsystem 24, and a dispenser assembly 26. The stand 22 includes a base 28 that is wheeled so as to maneuver the surgical sponge management system 20 within a medical facility. The stand 22 may include a main support 30 coupled to and extending upwardly from the base 28. The dispenser assembly 26 may be supported atop the main support 30 from which sponge sorters 32 may be suspended from articulating arms. The electronics subsystem 24 includes a module base 34, a user interface 36, and a data reader 38. The module base 34 may be secured to the stand 22. A mount removably couples the user interface 36 to the module base 34 or the stand 22. The user interface 36 (and/or the module base 34) may include one or more processors 42, memory, communications device, and/or other hardware. For example, the user interface 36 may be a tablet with a touchscreen display. The system 20 includes an optical sensor 44 (e.g., a color camera), and optionally may include a depth sensor 46 (e.g., an infrared camera), often collectively referred to herein as a camera 48. The camera 48 may be integrated on or removably coupled to the user interface 36, or may be standalone and in wired or wireless communication with the processor 42. In instances in which the camera 48 is integrated on a tablet, the tablet may be oriented upside down within the mount for the camera 48 to be positioned on a lower-front side of the tablet. The arrangement provides better alignment in elevation for users comfortably positioning the surgical sponge in front of their body to be detected by the camera 48 (see FIGS. 8-12). Further, neural networks to be described may be executed on the processor 42 of the tablet, on one or more processors of hardware coupled to or remote from the tablet, via cloud computing, combinations thereof, or the like.

The data reader 38 may be used either as a handheld device, or when supported by a cradle as shown in FIG. 1. The data reader 38 is configured to detect tags (T) associated with the surgical sponges(S). In an exemplary implementation, the data reader 38 is a wireless scanner, namely an RFID reader configured to detect RFID tags such as those disclosed in commonly-owned International Publication No 2021/041795, published Mar. 4, 2021, and International Publication No 2021/097197, published May 20, 2021, the entire contents of which being hereby incorporated by reference. Identifiers other than RFID tags are also contemplated, including optical tags (e.g., barcodes and quick response (QR) codes), and those disclosed in commonly-owned International Publication No. 2017/112051, published Jun. 29, 2017, the entire contents being hereby incorporated by reference. In implementations for use with surgical articles other than surgical sponges, the identifier may be the aforementioned tags, or ink printed on the surgical article.

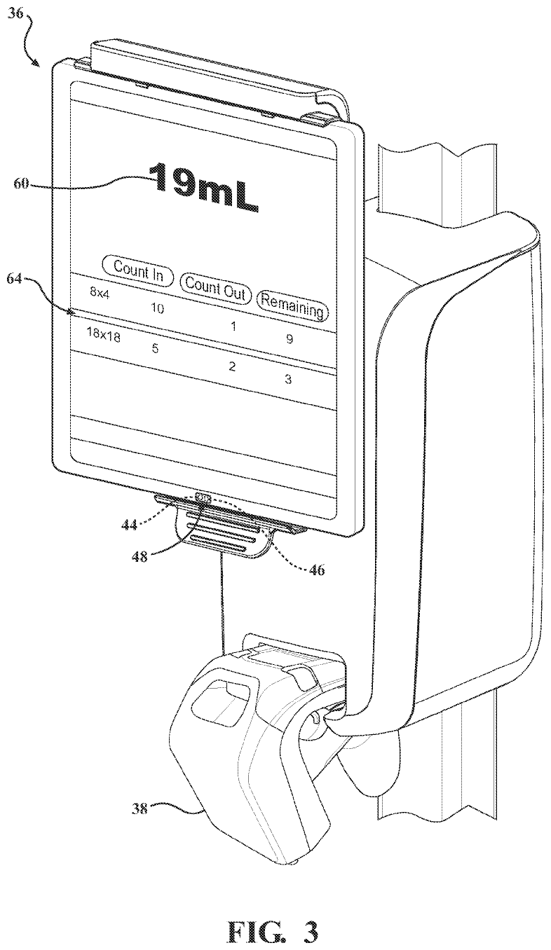

In a typical surgical procedure in which surgical sponges are utilized, the surgical sponges are counted in to be used during the surgical procedure. The surgical sponges may be counted in by causing the tag to be detected by the data reader 38. For example, the user may position the surgical sponge (or a bundle of the surgical sponges) near the data reader 38. The detected tag transmits identifying data to the processor 42, which includes at least one unique identifier indicative of characteristics of the surgical sponge. The unique identifier may include sponge size or type (e.g., 2×16 gauze, 4×4 gauze, 4×8 gauze, 4×12 sponge, 12×12 sponge, and 18×18 laparotomy sponge, etc.), material construction (e.g., four ply, eight ply, sixteen ply, etc.), absorbency, stretchability, dry weight, and the like. The processor 42 is configured to determine a type of the surgical sponge based on the unique identifier(s). Further, based on the data reader 38 detecting the tag, the processor 42 may index a counter 64 to reflect the one or more surgical sponges being counted in to surgical procedure. The counter 64 may be displayed on the user interface 36 (see FIG. 3). Either during or after the surgical procedure, the surgical sponges—used and unused—are typically counted out. The tags of the surgical sponges are again positioned to be detected by data reader 38 to identify the surgical sponges as being counted out. The processor 42 may index the counter 64 accordingly (e.g., subtract by one from the previous quantity of sponges counted in), and display, on the user interface 36, the quantity of the surgical sponges that remain counted in. The counter 64 may also display the quantity of the surgical sponges counted in originally, and/or the quantity of the surgical sponges that have already been counted out. In practice, the user, after confirming on the user interface 36 that the surgical sponge has been successfully counted out, immediately places the surgical sponge in one of the pockets of the sponge sorter 32.

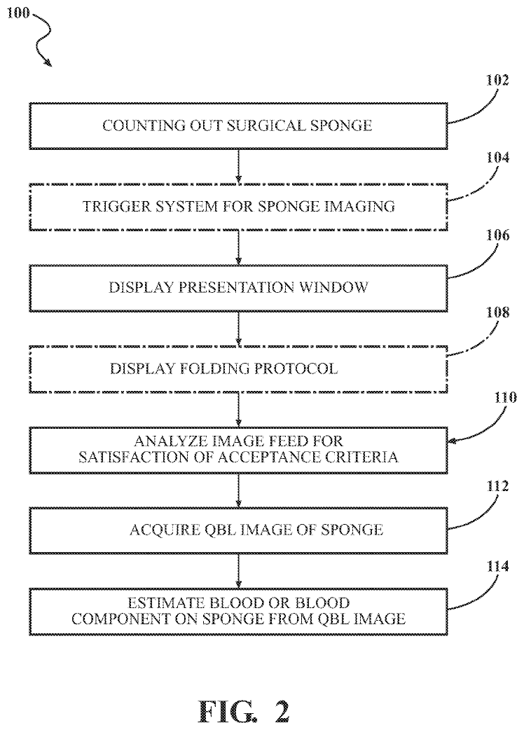

The system 20 and methods 100, 200 of the present disclosure facilitate quick and accurate assessment of fluids contained on the surgical sponges, and in particular blood. The relevant steps may be performed in just moments between the step of counting out the surgical sponge and placing the surgical sponge in the sponge sorter 32, and therefore with minimal disruption to the surgical workflow. Alternatively, the steps may be performed at other points in surgical procedure as desired by the user. Referring now to FIG. 2, the method 100 of assessing the surgical sponges bloodied in the surgical procedure is provided. The method 100 includes counting out the surgical sponge (step 102). As mentioned, the tag of the surgical sponge is detected by the data reader 38, and the data contained therein is transmitted from the data reader 38 to the processor 42. In particular, the unique identifier(s) stored on the tag are received by the processor 42. The processor 42 identifies the type of the surgical sponge from a database of predetermined sponge types based on the unique identifier. For example, the processor 42 may identify the surgical sponge as an 18×18 laparotomy sponge. The sponge type may be prompted or populated on the user interface 36 (e.g., a “pop up”), and optionally with an audible indicator, to indicate a successful scan of the sponge for the user to quickly ascertain the sponge type having been counted out. The user interface 36 may index and display the counter 64 including an updated count for each sponge type that has been counted in to and counted out of the surgical procedure, as shown in FIG. 3. Certain guardrail data to be described is also based on (e.g., unique to) the sponge type being counted out, and the data is transmitted, loaded, or otherwise readied by the processor 42 for the image-based processing.

The method may include triggering the system 20 for sponge imaging (step 104). The step may include automatically activating the camera 48 in response to the detection of the tag with the data reader 38. The step of automatic activation is optional, and it is alternatively contemplated that the user may provide an input to the user interface 36 to activate the camera 48 to perform the subsequent steps of the method 100. Alternatively, a mode may be selected on the user interface 36 prior to commencement of the surgical procedure for procedure types in which QBL protocols (e.g., the methods 100, 200) are to be utilized, after which the camera 48 is automatically activated in response to the detection of the tag with the data reader 38.

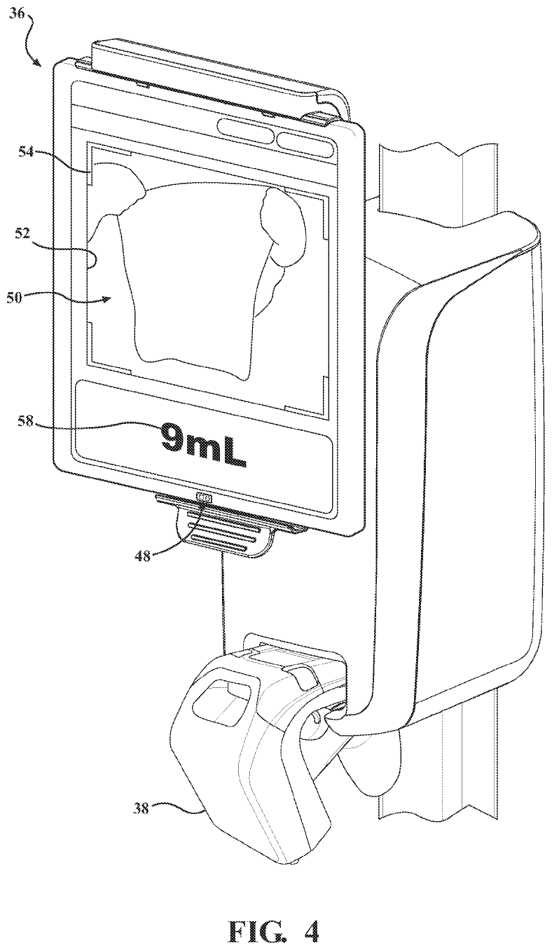

The step may further include displaying, on the user interface 36, an image feed 50 as detected by the camera 48. The image feed 50 may also be considered a sponge capture screen. The image feed 50 may be based on data being received from the optical sensor 44 and/or the depth sensor 46. FIG. 4 illustrates an example of the image feed 50 in which image frames form a video of the user holding the sponge in a field of view 52 of the optical sensor 44 is shown. The image feed 50 may also include graphical augmentations or indicia based on data from the optical sensor 44 and/or the depth sensor 46. One example includes a presentation window 54 overlaying the image feed 50, and another example includes the image feed 50 being in greyscale (based on depth data) and a segmentation mask 72 of the detected sponge being highlighted as green. The user may be trained to “present” the surgical sponge to the camera 48, preferably immediately after counting out the surgical sponge. The user generally grasps or pinches the surgical sponge by the upper corners to expose the largest area of the surgical sponge to the camera 48, and instructions to this effect may be provided on the user interface 36. The forward-facing orientation of the camera 48 and the display of the user interface 36 permits the user to utilize the image feed 50 to assist with real-time alignment of the surgical sponge in manners to be described.

The displaying of the image feed 50 may be automatically performed in response to the detection of the tag with the data reader 38. Therefore, the camera 48 may be automatically activated, and the display of the user interface 36 automatically updated to include the image feed 50. As generally appreciated from FIG. 4, the counter 64 has been replaced with the image feed 50, and certain other fields of the display have been shifted, resized, or removed. In such a manner, the image feed 50 is sized to be easily visible by the user standing at the acceptable presentation distance from the camera 48.

The method 100 includes displaying, on the user interface 36, the presentation window 54 overlying the image feed 50 being captured by the camera 48 (step 106). The presentation window 54 is visual indicia to guide the user to present the surgical sponge in the appropriate manner and at the appropriate distance from the camera 48. For some sponge types, the distance is between eighteen and thirty-six inches, for example. With continued reference to FIG. 4, the presentation window 54 is depicted as four “corner brackets” representative of the four corners of the presentation window 54, but the presentation window 54 need not assume any particular geometric shape. The dimensions of the presentation window 54 are based on the identified type of the surgical sponge corresponding to the detected tag of the surgical sponge being counted out. The processor 42 may be further configured to determine acceptance criteria 74 based on the type of surgical sponge—and the presentation window 54 may be sized and scaled so that at least some of the acceptance criteria 74 are met if the user presents the surgical sponge in a manner to at least substantially match or conform to the presentation window 54. The step is optional, in which case the processor 42 may determine a dynamic presentation window based on an actual location of the sponge within the optical and/or depth sensors. The processor 42 may assess the acceptance criteria dynamically based on the dynamic presentation window.

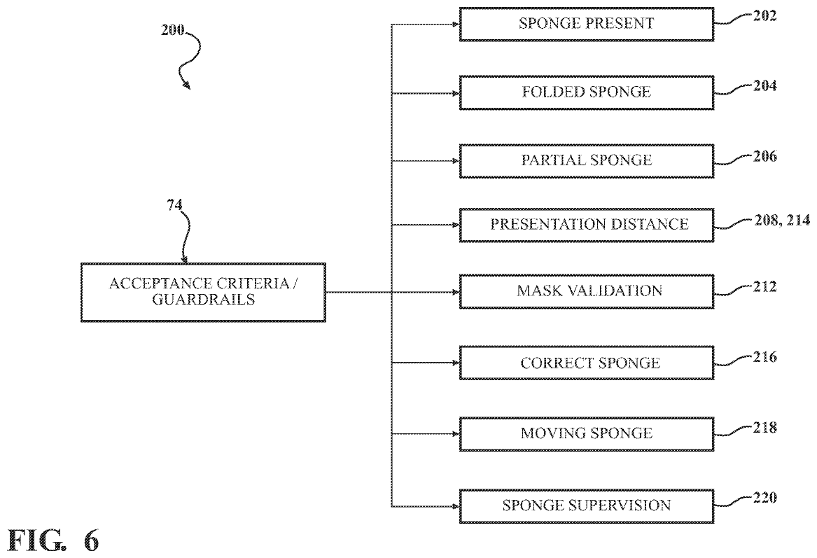

The method 100 includes a sponge recognition algorithm analyzing the image feed 50 for satisfaction of the acceptance criteria 74 (step 110). The step includes analyzing each image frame (or every few image frames) of the image feed 50 to ensure a full or stretched, correct surgical sponge is presented at the appropriate distance, unfolded, and not moving, among other acceptance criteria 74, also referred to herein as guardrails (see FIG. 6). If any one or more of the acceptance criteria 74 remains unsatisfied, the processor 42 prevents the camera 48 from capturing at least one QBL image of the surgical sponge for QBL analysis. As a result, characteristics of the QBL image of the surgical sponges captured by the optical sensor 44 are highly consistent—other than the blood content thereon—such that the sponge recognition algorithm trained to analyze the captured QBL images is correspondingly highly accurate.

Once the acceptance criteria 74 is determined to be satisfied, the method 100 includes the step of acquiring, with the camera 48, the QBL image(s) of the surgical sponge (step 112), also referred to herein as “sponge scans.” In one example, the QBL image(s) may be the subsequent frame(s) of the image feed 50 immediately following satisfaction of the acceptance criteria 74 analyzed by the sponge recognition algorithm, including output from a sponge recognition state machine (SRSM) 73. The QBL images may be considered distinct from the image frames from the image feed 50 that are being analyzed for satisfaction of the acceptance criteria. Rather, the QBL image is analyzed with a hemoglobin estimation algorithm for determination of a blood component (e.g., hemoglobin) of the liquid on the surgical sponge (step 114), for example, in the manner disclosed in the aformentioned U.S. Pat. Nos. 8,897,523 and 10,424,060. In particular, the processor 42 may be configured to extract a color component value (e.g., a redness value) from the QBL image, and execute the hemoglobin estimation algorithm to determine the hemoglobin concentration on a pixel-by-pixel or other suitable basis. The hemoglobin estimation algorithm may be unique to the sponge type having been counted out. In other words, hemoglobin estimation algorithm may be trained on extensive datasets for each sponge type to be compatible with the system 20. Blood loss may be determined based on the blood component, and a pixel-based or real-world area of the surgical sponge.

The user interface 36 may provide a prompt—e.g., visual and/or audible indicia—to notify the user of a successful QBL image capture, and immediately present an item-specific blood loss metric 58 for that particular sponge. For example, FIG. 4 is representative of the surgical sponge having absorbed nine milliliters or blood. The user deposits the surgical sponge into the sponge sorter 32. Further, the user interface 36 may be updated in real time to reflect a cumulative blood loss of the patient. The user interface 36 may—after a brief delay (e.g., one or two seconds)—return to the counter 64 (see FIG. 3), wherein a cumulative blood loss metric 60 is updated along with the sponge count being updated.

By using the sponge type to determine characteristics of the presentation window 54 and the acceptance criteria 74, the image-based QBL may be applied to more or most types of surgical textiles, including those of differing sizes, plys, and/or colors. By contrast, for example, should the system not know the sponge type (and thus its size), a smaller sponge type imaged at a close distance may be mistaken for a larger sponge type presented at a greater distance, or a larger sponge type imaged at a closer distance may have its outer portions truncated. Likewise, differing number of plys cause images of a different redness values, especially in instances in which it is imaged in a folded state. The system 20 and methods of the present disclosure overcomes such shortcomings by determining blood loss on surgical sponges of differing types in a seamless manner within the surgical procedure without needing to predefine the type of surgical sponge being used or counted out.

For certain surgical sponges, the presentation window 54 being displayed on the user interface 36 may require the user to fold the surgical sponge. One instance may include larger or elongate surgical sponges that, if presented unfolded, would require the user to be too far away from the camera 48 for accurate analysis. Moreover, requiring the folding of certain surgical sponges results in the dimensions of respective presentation windows 54 associated with different sponge types to be sufficiently different. Consequently, the acceptance criteria 74 are sufficiently different for each type of surgical sponge such that the user is generally prevented from satisfying the acceptance criteria 74 with an incorrect sponge type or an incorrect folding of the sponge.

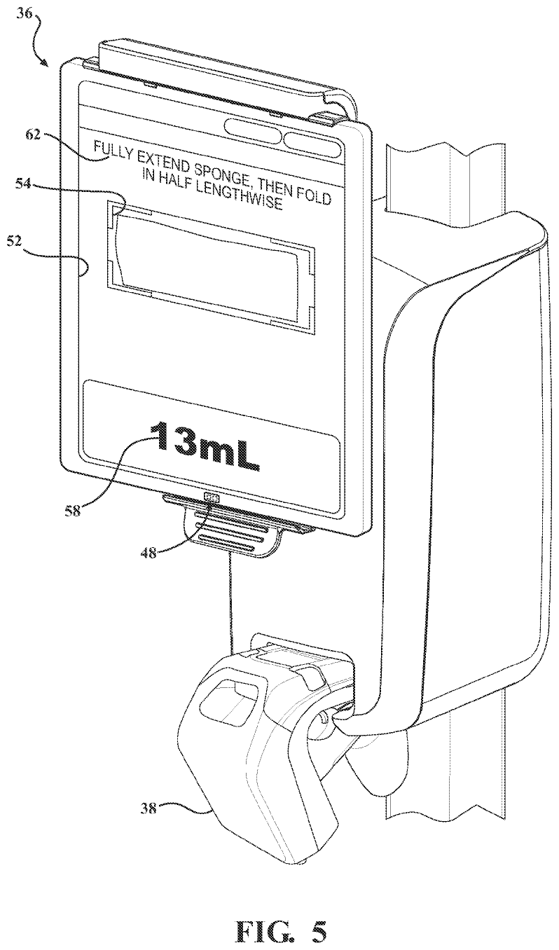

In certain implementations, the method 100 may include the step of displaying, on the user interface 36, a folding protocol to the user (step 108). The step is optional, and alternatively the presentation window 54 may require the surgical sponge be presented in an unfolded configuration. The folding protocol may include at least two aspects. First, the presentation window 54 is dimensioned to approximate the dimensions of the surgical sponge when folded according to the folding protocol. In other words, the presentation window 54 may be too small for the surgical sponge if presented unfolded. Second, the folding protocol may include guidance 62 to facilitate the user folding the surgical sponge in the desired manner. The guidance 62 may be textual instructions or graphical indicia, as shown in FIG. 5, and/or audible content. In addition to a folding protocol, it is contemplated that the system 20 may require the user to image both sides of the folded surgical sponge.

An exemplary workflow is described with reference to FIGS. 3-5. The counter 64 displayed in FIG. 3 shows two types of surgical sponges having been counted into the surgical procedure—4×8 gauzes, and 18×18 laparotomy sponges. The user counts out one of the 18×18 laparotomy sponges. The user interface 36 refreshes or updates to include the image feed 50, and the presentation window 54 corresponding to the 18×18 laparotomy sponge, as shown in FIG. 4. The presentation window 54 is a square that is sized to the field of view 52 of the camera 48. The square-shaped presentation window for the square-shaped surgical sponge (and with no further guidance being provided) indicates the user is to present the surgical sponge in the unfolded configuration. Once the user aligns the unfolded 18×18 laparotomy sponge to approximate the presentation window 54, the acceptance criteria 74 are likely to be satisfied and the QBL image is captured by the camera 48. The hemoglobin estimation algorithm unique to the 18×18 laparotomy sponge is applied, and the blood loss on the sponge is determined. At a later point in the surgical procedure, the user counts out one of the 4×8 gauzes. The user interface 36 again refreshes or updates to include the image feed 50, and displays the presentation window 54 corresponding to the 4×8 gauze. As appreciated from FIG. 5, the presentation window 54 is a rectangle that is sized appreciably smaller than the field of view 52 of the camera 48. The user interface 36 also includes the guidance 62, namely textual instructions to manipulate the surgical sponge in a specified manner (i.e., the folding protocol). Doing so results in the size of the folded 4×8 gauze approximating the size and shape of the presentation window 54 being displayed. Presenting the surgical sponge in a manner other than as required by the guidance 62 effectively prevents the acceptance criteria 74 for the 4×8 gauze to be satisfied, and thus the camera 48 being prevented from capturing the QBL image. Once the folded 4×8 is presented in a manner that approximates the presentation window 54, the acceptance criteria 74 are likely to be satisfied and the QBL image is captured by the camera 48. The hemoglobin estimation algorithm unique to the 4×8 gauze sponge is applied, and the blood loss on the sponge is determined. The item-specific blood loss metric 58 is displayed, in this case thirteen milliliters of blood. It should be appreciated that the methods 100, 200 disclosed herein may be performed in mere seconds without noticeable disruption of workflow and without requiring the user to select the sponge type, and/or to manually trigger the sponge scan of the presented surgical sponge within the field of view 52 of the camera 48.

As mentioned, the one or more processors 42 implement or execute the sponge recognition algorithm. The sponge recognition includes neural networks are configured to process the images of the surgical sponges to detect the sponge and apply the acceptance criteria 74 or guardrails. In an exemplary implementation, there are two neural networks: a localization neural network 66 and a segmentation neural network 68. The localization neural network 66 may be a deep neural network such as a convolutional neural network (e.g., ResNet-50, MobileNet-v1,2 etc.), a transformer-based neural network (e.g., ViT), or a combination thereof. The output of the localization neural network 66 is modified to have both a classification head as well as a regression head. The localization neural network 66 is configured to, among other actions, determine an image class: whether or not a sponge is being presented (sponge precent), is folded or crumbled (folded sponge), is only partially visible (partial sponge). The localization neural network 66 is further configured to provide a bounding box 70 around the detected sponge (see FIGS. 10-12). The bounding box 70 may include a width (w), height (h), and offset (x, y).

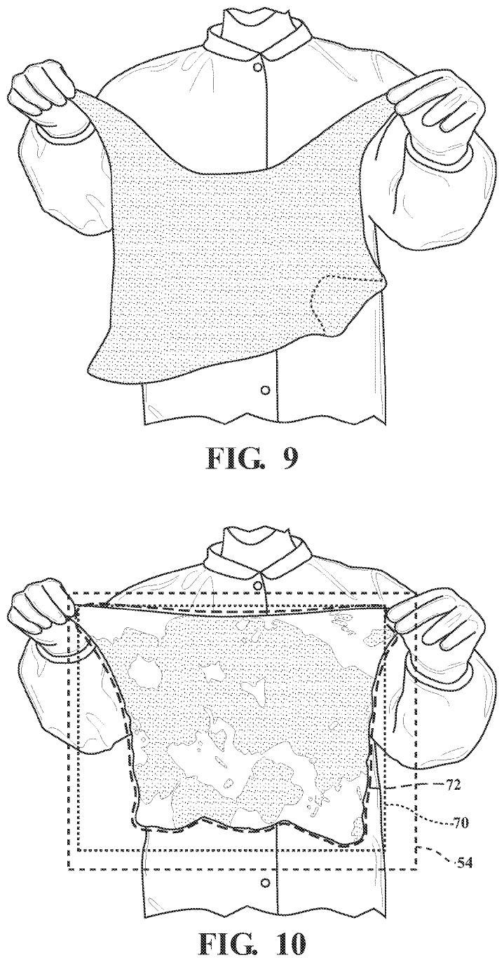

The segmentation neural network 68 may be an encoder-decoder style convolutional neural network architecture such as a U-Net, Mask-R CNN, DeepLab, or transformer-based neural network architectures that support image segmentation. The output of the segmentation neural network 68 provides per-pixel prediction of a segmentation mask 72 (schematically represented in FIGS. 9-11) of whether a pixel is sponge pixel or background pixel. In an exemplary implementation, the segmentation neural network 68 may utilize data from both the optical sensor 44 and the depth sensor 46. Alternatively, the segmentation neural network 68 may provide the segmentation mask 72 based only on the color image. The segmentation neural network 68 is configured to, among other actions to be described, provide the sponge segmentation mask 72 and facilitate determinations involving the acceptance criteria 74.

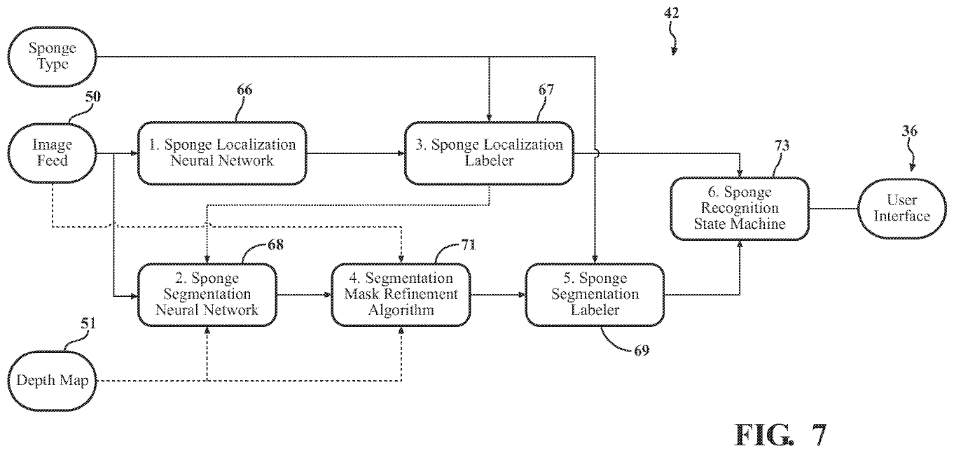

As reflected in FIG. 7, the sponge recognition algorithm includes a localization labeler 67, a segmentation labeler 69, and the sponge recognition state machine (SRSM) 73. The localization labeler 67 and the segmentation labeler 68 are each configured to generate classification labels—namely a localization label and a segmentation label—based on respective analyses of certain acceptance criteria 74. The localization and segmentation labels are fed to sponge recognition state machine (SRSM) 73. In particular, based on the labels of per-image predictions, the SRSM identifies video-based states. The SRSM uses dominant class predictions over a preset number of previous frames to ensures that the video-based states are correct even when a single frame may be incorrectly classified.

The localization neural network 66 may run in real time on slower devices and facilitate real-time user interactions, whereas the segmentation neural network 68 may be considered a larger and more accurate convolutional neural network. The dual-mode design ensures real-time processing of the image frames of the image feed 50 without compromising on the accuracy of the sponge segmentation mask 72. Alternatively, both the neural networks 66, 68 can be combined into a single hybrid network that outputs both the classification labels, the segmentation mask 72, and the bounding box 70. Such a hybrid neural network may run in real-time on faster devices having sufficient graphics processing capabilities. The neural networks may be trained in manners disclosed herein in order to perform the methods of the present disclosure with the necessary accuracy. The output of the sponge recognition algorithm is provided to the hemoglobin estimation algorithms that are unique to the sponge type being counted out.

Referring now to FIGS. 6-8B, a method 200 of assessing each of the acceptance criteria 74 or guardrails is described in turn. The method 200 includes executing algorithms by at least one of the neural networks 66, 68. The acceptance criteria 74 may be assessed in the order depicted, or in any suitable order unless otherwise specified. First, the method 200 includes determining whether a surgical sponge is present (step 202). The localization neural network 66 determines whether or not a surgical sponge is present in the image frames of the image feed 50. For example, the surgical sponge may not yet have been brought into the field of view 52 of the camera 48, or it is too far from the camera 48 to be detected. If no sponge is detected, the acceptance criteria 74 is identified to be not satisfied.

If the surgical sponge is detected in step 202, the method 200 includes determining whether the surgical sponge is folded (step 204). FIG. 9 shows a common manner by which the sponge is often held improperly. In particular, the user may not provide sufficient tension along the upper aspect of the surgical sponge such that the slack causes the upper aspect to droop. Further, in this instance a portion of the lower corner of the surgical sponge is folded back on itself. The localization neural network 66 may be trained to recognize folded, slacked, or crumbled sponge images, and classify as “folded sponge.” The step 204 of determining whether the sponge is folded may also include determining whether the surgical sponge is at least substantially square or rectangular. Referring to FIG. 10, the localization neural network 66 is configured to, among other actions, provide the bounding box 70 around the detected sponge. The bounding box may include information about the detected sponge, for example, a width, height, x-shift and y-shift. Based on outer edges of the bounding box, the localization labeler determines whether the surgical sponge is sufficiently square or rectangular. The determination may be associated with the sponge type. The extent by which the surgical sponge is square or rectangular to satisfy the guardrail may be based on the localization neural network 66 being trained on datasets of folded and correctly held sponges. For example, if a 18×18 laparotomy sponge is counted out, the localization neural network 66 may determine whether the presented surgical sponge is sufficiently square. If an 4×12 gauze is counted out (and to be presented in the unfolded configuration), the localization neural network 66 may determine whether the presented surgical sponge is sufficiently rectangular.

The step 204 of determining whether the sponge is folded may further include determining whether an aspect ratio of the presented surgical sponge is within an acceptable range based on the sponge type being counted out. As mentioned, the bounding box 70 includes a width and a height-a ratio of the two being the determined aspect ratio. If the sponge is determined to be sufficiently square or rectangular and yet its determined aspect ratio is outside the acceptable range of the aspect ratio for that sponge type, the localization neural network 66 determines the sponge is a folded sponge (step 204). For example, for the 18×18 laparotomy sponge, its manufactured aspect ratio may be approximately 1.0. Owing to the training of the localization neural network 66 on the datasets of 18×18 laparotomy sponges being presented by users in varying manners, the algorithm implementing the guardrail may have established the acceptable range of the aspect ratio to be between, for example, 0.7 and 1.5. These variances account for instances where the user may stretch the sponge, stretchability of the sponge material, minor warping while being held from the upper corners, and the like. If the user counts out an 18×18 laparotomy sponge and presents the sponge in a manner where its determined aspect ratio is outside the acceptable range, the localization neural network 66 may determine that the 18×18 laparotomy sponge is folded or in a folded condition. Additionally or alternatively, the determined aspect ratio falling outside of the acceptable range may be indicative that a wrong sponge is being presented. If a folded sponge (or a wrong sponge) is detected, the acceptance criteria 74 is identified to be not satisfied. The localization neural network 66 generates a localization label the presented surgical sponge is a folded sponge.

As mentioned, requiring the folding of certain surgical sponges also results in the dimensions of the respective presentation windows 54 for different sponge types to be sufficiently different. By extension, this applies to the acceptable ranges of the aspect ratios. In other words, the acceptable ranges of the aspect ratios are sufficiently different for each type of surgical sponge such that the user is generally prevented from satisfying the step 204 with an incorrect sponge type. For example, the acceptable range for a 4×8 gauze, if folded according to the folding protocol, may be between 1.7 and 2.2. A compatible sponge type immediately smaller—e.g., a 2×16 gauze, if folded according to the folding protocol, if necessary—may be associated with an acceptable range between 4.9 and 7.8. Likewise, a compatible sponge type immediately larger—e.g., a 4×12 gauze, if folded according to the folding protocol, if necessary—may be associated with the acceptable range between 2.5 and 3.4. While these values are merely exemplary, it should be appreciated that none of the acceptable ranges overlap between the 2×16 gauze, the 4×8 gauze, and the 4×12 gauze. For square-shaped sponges—e.g., 12×12 sponge and 18×18 laparotomy sponge—the acceptable ranges may overlap; however, a correct sponge guardrail (step 216) to be described addresses such considerations.

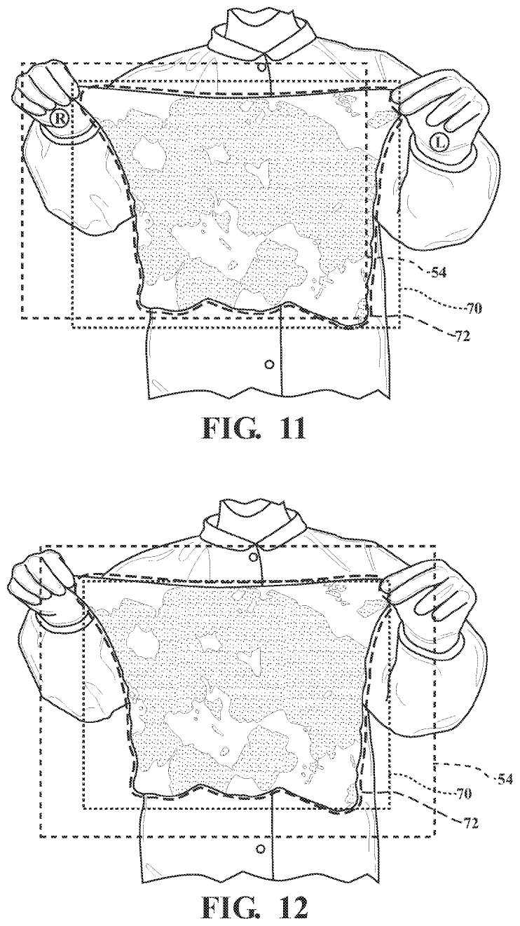

The method 200 may include determining whether the sponge being presented is a partial sponge by being too close to a boundary (step 206). If the segmented representation of the presented surgical sponge extends beyond the field of view 52 of the camera 48, the localization neural network 66 determines a partial sponge condition. For example, if an outermost pixel of the field of view 52 of the camera 48 is determined to be a sponge pixel, and/or if the bounding box 70 is determined to extend through outermost pixel, the localization neural network 66 determines the partial sponge condition. With concurrent reference to FIG. 11, the localization labeler 66 may determine whether the bounding box 70 is too close to the presentation window 54. In particular, the localization labeler 66 determines a margin, i.e., a pixel-based distance between the bounding box 70 and the presentation window 54. The training of the localization labeler 66 on the datasets may establish margin threshold values associated each of the top, bottom, left, and right of the presentation window 54. Alternatively, the margin threshold values may be inputted into the software. In one example, the margin threshold values may be set to zero such that, if the bounding box 70 intersects with the presentation window 54, the localization neural network 66 determines the partial sponge condition. FIG. 11 shows the left side (based on the user's left hand (L)) of the bounding box 70 passing through the presentation window 54, resulting in a negative value for the margin. Likewise, the bottom of the bounding box 70 passes through the presentation window 54. Collectively, the user in FIG. 11 is presenting the sponge too far left and too low to satisfy the acceptance criteria 74. The localization labeler 67 generates the localization label that the presented surgical sponge is a partial sponge. Of course, the image feed 50—including the presentation window 54—is presented on the user interface 36 such that the user can easily adjust the position of the presented surgical sponge. A prompt or indicia may be provided on the user interface 36 to guide the user's movement accordingly.

The localization neural network 66 and the localization labeler 67 may be further configured to determine whether the sponge is positioned too close or too far from the camera 48 by determining whether the sponge is too large or too small, respectively (step 208). In particular, the localization labeler 67 is configured to determine a pixel-based area of the bounding box 70, and compare the bounding box area against an acceptable range of bounding box area that is unique to each sponge type. The localization labeler 67 may be trained on the datasets for the algorithm to establish the acceptable ranges of bounding box areas, or the acceptable ranges may be inputted or otherwise determined. In one example, the acceptable range of the bounding box area approximates the presentation window 54 being displayed on the user interface 36, which again is unique to the sponge type being counted out. The acceptable ranges of bounding box areas are pixel-based such that the steps 208 may be based on only the optical image from the optical sensor 44. If the processor 42 determines the bounding box area is greater than or less than the acceptable range of the bounding box area, the localization labeler 67 generates the localization label that the sponge is too close or too far (from the camera 48), respectively. FIG. 12 shows the bounding box 70 having an area appreciably smaller than an area of the presentation window 54 such that, for example, the presented surgical sponge may be identified as being too far from the camera 48. Again, the image feed 50 is presented on the user interface 36 such that the user may adjust the position of the sponge, and corresponding prompts may be provided on the user interface 36 to guide the user's movement.

If the localization neural network 66 determines the sponge to be presented as unfolded and otherwise appropriately positioned within the presentation window 54, the method 200 includes identifying the presented surgical sponge as a full sponge (step 210), and the localization labeler 67 generates the localization label accordingly for further processing by the SRSM 73.

With the processing by the localization neural network 66, the segmentation neural network 68 also processes the image frames of the image feed 50. The dual-mode processing may occur concurrently or sequentially. The segmentation neutral network 68 outputs the segmentation mask 72 in which sponge pixels and the background pixels are separated. FIGS. 9-11 represent the segmentation mask 72 conforming to the presented surgical sponge in a highly accurate manner; e.g., pixel-by-pixel. Further, the segmentation neutral network 68 may utilize a depth map 51 of depth data obtained from the depth sensor 46. A segmentation mask refinement algorithm 71 may reject pixels from the segmentation mask 72 that have different depth values that exceed a threshold when compared to most of the mask pixels. For example, differences in the depth values may be estimated as the distance between respective points and plane fitted to the depth map. The threshold may be the same or different for different sponge types. Further, the depth map 51 may be processed to determine real-world dimensions of the presented surgical sponge for assessing further acceptance criteria 74, including presentation distance (step 214), correct sponge confirmation (step 216), unmoving sponge confirmation (step 218), and sponge supervision (step 220).

Referring to FIGS. 6 and 8B, the segmentation neutral network 68 may run on the image frames of the image feed 50 only for which the localization neural network 66 has determined a sponge is present (see step 202). Otherwise, the segmentation mask 72 may be identified as not available or empty. If the sponge segmentation mask 72 is determined to not be empty, the method 200 includes validating the sponge segmentation mask 72 (step 212). The step 212 may include determining number of pixels in the sponge segmentation mask 72, and comparing the determined number of pixels against an acceptable range of pixels. The acceptable range of pixels is based on the sponge type, which again is identified by the processor 42 with the counting out of with the data reader 38. If the segmentation mask 72, as determined by the segmentation neutral network 68, does not have a sufficient number of pixels, the segmentation mask 72 is determined to be invalid.

The depth data may be used to establish thresholds on acceptable presentation distance; i.e., lower and upper limits on real-world distance by which the user must present the surgical sponge from the camera 48. The method 200 may include determining whether the presented surgical sponge is too close or too far (step 214). The segmentation labeler 69 is configured to output that the sponge is either too close or too far if the average depth of all pixels belonging to the sponge segmentation mask 72 is less than or greater than the minimum threshold or maximum threshold, respectively. As previously described, the localization neural network 66 does a similar process by which the size of the bounding box 70 is compared to the size of the presentation window 54 (see step 208), which is not reliant on the depth data or real-world dimensions.

Further, the real-world area of sponge being imaged may be determined if depth map 51 is available. The segmentation labeler 69 does so by determining a sponge area (of the segmentation mask 72) based on the depth map of the image frames of the image feed 50. The determined sponge area is compared against an acceptable range of sponge area. The determined sponge area may be computed as a squared root L2-squared norm of all values from the depth map corresponding to the pixels of the image frame. The training of the segmentation labeler 69 on the dataset may establish the minimum and the maximum area for each type of the surgical sponges, or the acceptance ranges may be inputted or otherwise determined.

The method 200 may further include determining whether the presented surgical sponge is the correct sponge (step 216). As mentioned, the extent by which the localization neural network 66 may determine a correct sponge is based on a comparison of the determined aspect ratio against the acceptance range of the aspect ratio. The segmentation neural network 68 may confirm the determination, and further account for scenarios in which certain sponge types may have acceptable ranges that overlap. By leveraging the depth map to determine the real-world area of the segmentation mask 72, the segmentation labeler 69 is capable of differentiating between square-shaped sponges of differing dimensions. The segmentation labeler 69 may be trained on the datasets to establish the acceptable range of sponge areas, or the acceptable ranges may be inputted or otherwise determined. For example, at the appropriate presentation distance, an upper sponge area threshold for a 12×12 sponge may be 14000 AU, whereas a lower sponge area threshold for an 18×18 laparotomy sponge may be 16000 AU. Consequently, if a user were to count out an 18×18 laparotomy sponge and present a 12×12 sponge in a manner to otherwise satisfy the acceptance criteria (e.g., conform to the presentation window 54), the segmentation labeler 69 would determine the determined sponge area of the presented surgical sponge to be too low for the distance at which it is being presented. The segmentation neural network 68 determines the acceptance criteria 74 to be not satisfied, and the segmentation label may be generated accordingly. Corresponding prompts may be provided on the user interface 36 to indicate to the user that a wrong sponge may be presented.

The step 216 of whether the presented surgical sponge is the correct sponge may also include comparing a sponge compactness against a predetermined compactness threshold. In some respects, the step of comparing the sponge compactness may again be considered a crosscheck between the segmentation neutral network 68 and the localization neural network 66, which also determines an area and the aspect ratio of the bounding box 70, as mentioned. The predetermined compactness threshold may be a fraction of the area of the bounding box 70 that must be exceeded by the determined sponge area of the segmentation mask 72 to be determined as correct. For example, the predetermined compactness threshold may be 50% such that, if the determined area of the segmentation mask 72 is less than 50% of the determined area of the bounding box 70, the acceptance criteria 74 is determined to be not satisfied. This may be indicative of the wrong sponge, or of an invalid mask (see step 218). A corresponding prompt may be provided on the user interface 36 to request the user to represent the surgical sponge to the camera 48.

The method 200 may further include determining whether the presented surgical sponge is moving (step 218). If the presented surgical sponge is moving too quickly, motion blur may result compromise accuracy of the QBL analysis. The step 218 may include determining a centroid of the sponge segmentation mask 72, and further determining an average motion magnitude of the centroid within a predetermined number of image frames of the image feed 50 (e.g., five consecutive image frames). The average motion magnitude may be computed as an L2-distance between consecutively estimated centroids. The step 218 may include comparing the average motion magnitude against a centroid change threshold, which is unique for each sponge type. The centroid change threshold may be based on training the segmentation labeler 69 with the extensive datasets, or inputted into the software. If the average motion magnitude is greater than the centroid change threshold, the segmentation labeler 69 determines the presented surgical sponge to be moving too quickly, and the acceptance criteria 74 to be not satisfied. An alternative implementation includes tracking motion of the bounding box 70 of the detected sponge across consecutive image frames of the image feed 50 against a threshold. A corresponding prompt may be provided on the user interface 36 to request the user to hold the sponge steady.

Another acceptance criteria 74 or guardrail of the method 200 may include performing sponge supervision (step 220). As mentioned, the processor 42 may be configured to extract a redness value from the bloodied sponge for determining the concentration of the blood component on a pixel-by-pixel or other suitable basis. The redness of the pixels of the image are affected by environmental lighting, such as the light variations in the medical facility. It is known to use a calibration placard for light normalization to account for linear variations in ambient light intensity and color temperature. While generally effective, light “bleed-through” from a directional light source through the back of the presented surgical sponge may cause underestimation in predictions of the blood component. The light bleed-through may be exacerbated by users who hold the sponge away from the body, thereby exposing a greater portion of the surgical sponge to light sources positioned behind the user within the medical facility.

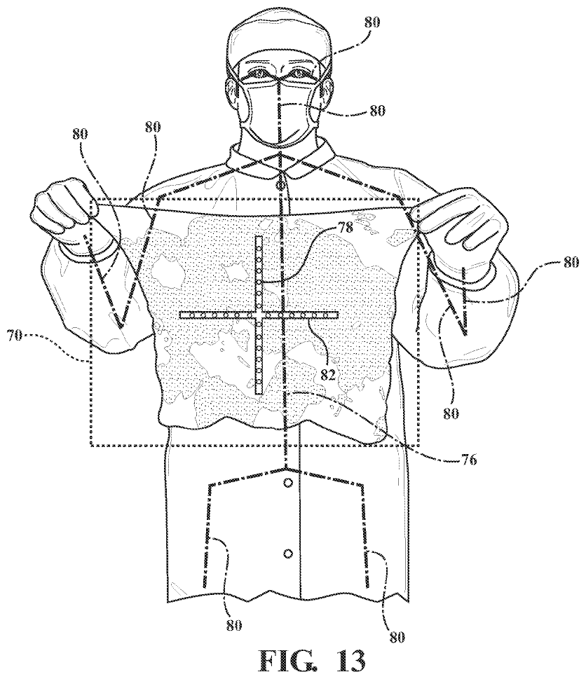

The step 220 of performing sponge supervision may include detecting the user's hand, torso, neck, and other associated body parts using a pose estimation neural network and a pose estimation labeler (not identified), as well as the location of the presented surgical sponge in manners previously described. Once the person and the presented surgical sponge are detected, the pose estimation labeler is configured to determine whether the presented surgical sponge is being held properly in front of the torso, which tends to limit light bleed-through. In particular and with reference to FIG. 13, the pose labeler is configured to identify a body centerline 76, and a sponge vertical centerline 78. Based on the identified centerlines 76, 78, the step 220 may include determining a horizontal shift in location of the presented surgical sponge relative to body centerline 76. Further, the pose estimation labeler may be configured to identify centerlines 80 or reference points of other anatomical landmarks, such as a head, neck, arms, hands, legs, or the like. One or more of the additional centerlines 80 may be compared against a horizontal sponge centerline 82 of the presented surgical sponge to determine a vertical shift of the presented surgical sponge. The horizontal shift and/or a vertical shift may be compared against a respective shift threshold, determined by the pose estimation algorithm trained on the datasets or inputted or otherwise determined. Guidance may be provided on the user interface 36 to instruct the user to present the surgical sponge in front of their torso if the shift(s) exceed the shift threshold. Moreover, the depth map from the depth sensor 46 may be used to distinguish between people in background and the foreground. Together with the location of the presented surgical sponge within the field of view 52 being known, the pose estimation algorithm is configured to determine which of multiple people in the field of view 52 of the camera 48 is presented the sponge.

If the segmentation neural network 68 determines the sponge to be presented at the appropriate distance and position relative to the user's body, the correct sponge, not moving, and otherwise that the segmentation mask 72 to be valid, the segmentation labeler 69 generates the segmentation label for further processing by the SRSM 73. This is in addition to the localization label generated by the localization labeler 67.

The SRSM is configured to receive the labels from each of the localization labeler 66 and the sponge segmentation labeler 68, and perform further processing prior to the camera 48 capturing the QBL image. The SRSM is configured to determine whether the localization and segmentation labels are equal. In other words, the SRSM determines whether the acceptance criteria 74 or guardrails, executed potentially separate by the localization neural network 66 and the sponge segmentation neural network 68, have been satisfied. With the image feed 50 being a video and the image frames effectively occurring in imperceptible rapid succession, image states of one or more of the acceptance criteria 74 may change outcome rapidly. The SRSM may execute a smoothing procedure to aggregate the localization and segmentation labels using a sliding window with an image frame size (L). The sliding window incorporates previous states between time “t−L” to time “t−1,” and may not include the most immediate state (at time t). The most frequent aggregated sponge label is called the dominant label. The SRSM may indicate the QBL image is ready for capture and analysis by the hemoglobin estimation engine if the required consecutive frames (K), including immediate and dominant labels, satisfy the acceptance criteria 74. As the number of required consecutive frames is increased, the probability of false positive scans decreases. The parameters L, K may be tuned based on empirical evaluations. If no dominant label is identified within the last K consecutive image frames, then the SRSM may generate an uncertain condition video state. For example, the uncertain condition video state may be generated if the most frequent aggregated sponge label in the sliding window occurs less frequently than half of the sliding window size, or other suitable value.

Once the QBL image is captured, the sponge recognition algorithm is configured to momentarily put the system 20 in a “cool down” state for the user to remove the presented surgical sponge from the field of view 52 of the camera 48. The SRSM may not permit further capturing of QBL images regardless of the immediate and dominant labels, at least until the dominant label identifies the sponge as no longer being or not present (see step 202) for a required number of consecutive image frames (M) of the image feed. The parameter M may be tuned based on empirical evaluations or otherwise determined. Once the sponge as no longer present for the required number of consecutive frames, the SRSM is configured to return to a video state in which the localization neural network 66 and segmentation neural network 68 is readied to perform the methods 100, 200 disclosed herein. In effect, this allows that only one sponge scan is captured per presented surgical sponge. The user is appropriately instructed to remove the sponge from the field of view 52 of the camera 48 before presented the next surgical sponge.

As mentioned, the neural networks 66, 68 may be trained using supervised, semi-supervised, and/or unsupervised learning methodology in order to perform the methods of the present disclosure, and in particular with the necessary accuracy. A diverse and representative set of training dataset is collected to train each of the neural networks on diverse scenarios both within and outside of expected uses of the system 20. Training methods may include data augmentation, pre-trained neural networks, and/or use semi-supervised learning methodology to reduce the requirement of labeled training dataset. The data augmentation may increase sample size such as lighting augmentation (brightness, contrast, hue-saturation shift, PlanckianJitter, etc.), geometric transformation (rotation, homography, flips etc.), noise and blur addition, custom augmentation (artificially enlarging or reducing the size of the sponge, cropping the sponge), or the like.

The datasets vary parameters including sponge size, sponge saturation, ground truth (blood color), lighting, imaging distance, sponge orientation, and position. For examples, the sponges may be saturated by varying levels of total value up to maximum blood-carrying capacity; imaging distance may vary from 0.5 to 1.5 meters; lighting may be brighter or lighter; sponge orientation may be rotated in plane or out of plane; position may be varied along with images of folded, unfolded sponges, and slightly folded. Still further, an example set of sponge-like non-sponge objects may be included in the datasets to train the neural networks to recognize white or red non-sponge objects as part of background.

Additional approaches include training the neural networks with the color image and the depth data together to jointly learn the segmentation mask using both the color image as well as depth image. Specific architectures such as ESANet can be used to efficiently combine the color image and depth data. The segmentation neural network 68 may also be trained for images where depth data is not available (e.g., only the color image). If the depth data is not available, a dataset may be generated that includes the pixel area within the segmentation mask being correlated at different distance of imaging. From a depth of a sponge scan from the color image, the segmentation mask, and sponge type being known, the segmentation neural network 68 is configured to determine whether the presented surgical sponge is scanned too close or too far from the system. Therefore, the segmentation neural network 68 is configured to provide the segmentation mask 72 based only the color image from the optical sensor 44. Lastly, for the pose estimation neural network of sponge supervision, exemplary models may include AlphaPose, MobileHumanPose, MoveNet, OpenPose, etc., which are further trained on an application-specific dataset, e.g., nurses imaging the sponge.

The foregoing disclosure is not intended to be exhaustive or limit the disclosure to any particular form. The terminology which has been used is intended to be in the nature of words of description rather than of limitation. Many modifications and variations are possible in light of the above teachings and the invention may be practiced otherwise than as specifically described.

Further inventive aspects of the present disclosure are disclosed with reference to the following exemplary clauses:

Clause 1—A method of assessing surgical articles with a system including a data reader, one or more processors, a user interface, an optical sensor, the method comprising: identifying, with the one or more processors, an article type of the surgical article from a database of predetermined types of surgical articles; detecting, with a field of view of the optical sensor, a presented surgical article; capturing at least one image of the presented surgical article; determining, with the one or more processors, whether the presented surgical sponge satisfies at least one acceptance criteria, wherein the acceptance criteria are based on the identified article type that was scanned with the data reader; and displaying, on the user interface, at least one of confirmation that the presented surgical article is of the same type that was scanned with the data reader, and information related to a state or condition of the surgical article.

Clause 2—The method of clause 1, wherein each of the surgical articles includes a tag storing a unique identifier, the method further comprising: detecting, with the data reader, the tag of the surgical article, wherein the unique identifier stored on the tag is received by the processor; and identifying, with the one or more processors, the article type based on the unique identifier.

Clause 3—The method of clause 1 or 2, wherein the step of determining whether characteristics of the surgical article satisfy the acceptance criteria comprises: generating, with the one or more processors, a segmentation mask of the presented surgical article; determining, with the one or more processors, a number of pixels in the segmentation mask; comparing, with the one or more processors, the determined number of pixels of the segmentation mask against a database images in which the surgical article is in an unused or undamaged condition; and displaying, on the user interface, the information indicative that the condition of the surgical article is damaged if the determined number of pixels is outside of an acceptable range of pixels.

Clause 4—The method of clause 3, wherein the system includes a depth sensor, and wherein step of determining whether characteristics of the surgical article satisfy the acceptance criteria comprises: determining, with the one or more processors, a real-world article area of the segmentation mask based on depth data from the depth sensor; comparing, with the one or more processors, the determined real-world sponge area of the segmentation mask against an acceptable range of article area, wherein the acceptable range of article area is based on the identified article type; identifying, with the one or more processors, the presented surgical article as a wrong article if the determined real-world sponge area of the segmentation mask is outside the acceptance range of the article area.

Clause 5—The method of any one of clauses 1-4, wherein the step of determining whether characteristics of the surgical article satisfy the acceptance criteria comprises: generating, with the one or more processors, a bounding box about or around of the presented surgical article; determining, with the one or more processors, an aspect ratio of the bounding box; and comparing, with the one or more processors, the determined aspect ratio of the bounding box against an acceptable range of the aspect ratio, wherein the acceptable range of the aspect ratio is based on the identified article type; identifying, with the one or more processors, the presented surgical article to be a wrong type of surgical article if the determined aspect ratio of the bounding box is not within the acceptable range of the aspect ratio.

Clause 6—The method of clause 5, wherein the step of determining whether characteristics of the surgical article satisfy the acceptance criteria comprises: determining, with the one or more processors, an area within the bounding box; comparing, with the one or more processors, the determined area of the bounding box against an acceptable range of bounding box area, wherein the acceptable range of the bounding box area is based on the identified article type; identifying, with the one or more processors, the presented surgical article to be too close or too far from the optical sensor if the determined area of the bounding box is outside the acceptable range of the bounding box area.

1. A method of assessing surgical sponges bloodied in a surgical procedure with a sponge management system including a data reader, one or more processors, a user interface, and an optical sensor, wherein each of the surgical sponges includes a tag storing a unique identifier, the method comprising:
detecting, with the data reader, the tag of the surgical sponge, wherein the unique identifier stored on the tag is received by the one or more processors;
identifying, with the one or more processors, a sponge type of the surgical sponge from a database of predetermined types of surgical sponges based on the unique identifier;
detecting, with a field of view of the optical sensor, a presented surgical sponge;
determining, with the one or more processors, whether the presented surgical sponge satisfies at least one acceptance criteria, wherein the acceptance criteria are based on the identified sponge type of the surgical sponge that was counted out;
capturing, with the optical sensor, a QBL image of the presented surgical sponge if the acceptance criteria are determined to be satisfied;
estimating, with the one or more processors, a volume of the blood or a blood component based on the QBL image; and
displaying, on the user interface, the volume of the blood or the blood component.

2. The method of claim 1, further comprising displaying, on the user interface, a presentation window overlying an image feed as being captured by the optical sensor, wherein dimensions of the presentation window are based on the identified sponge type of the surgical sponge.

3. The method of claim 2, further comprising providing, on the user interface, instructions to manipulate the presented surgical sponge according to a folding protocol based on the identified sponge type, wherein the dimensions of the presentation window are further based on dimensions of the surgical sponge following the folding protocol.

4. The method of claim 2, wherein the step of determining whether characteristics of the surgical sponge satisfy the acceptance criteria comprises:
generating, with the one or more processors, labels indicating whether a sponge-like object is presented in a stretched configuration fully inside the presentation window, and a bounding box about or around of the presented surgical sponge; and
determining, with one or more processors, whether the bounding box of the presented surgical sponge is square or rectangular within a predetermined geometric threshold.

5. The method of claim 4, wherein the step of determining whether characteristics of the surgical sponge satisfy the acceptance criteria further comprises:
determining, with the one or more processors, an aspect ratio of the bounding box; and
comparing, with the one or more processors, the determined aspect ratio of the bounding box against an acceptable range of the aspect ratio, wherein the acceptable range of the aspect ratio is based on the identified sponge type of the surgical sponge;
identifying, with the one or more processors, the presented surgical sponge to be in a folded condition if the determined aspect ratio of the bounding box is not within the acceptable range of the aspect ratio; and
preventing the optical sensor from capturing of a QBL image sponge of the presented surgical sponge in the folded condition.

6. The method of claim 4, wherein the step of determining whether characteristics of the surgical sponge satisfy the acceptance criteria comprises:
determining, with the one or more processors, a margin between the bounding box and the presentation window;
comparing, with the one or more processors, the margin against a margin threshold value;
identifying, with the one or more processors, the presented surgical sponge to be a partial sponge if the determined margin is less than the margin threshold value; and
preventing the optical sensor from capturing of a QBL image sponge of the partial sponge.

7. The method of claim 6, wherein the determined margin is a pixel-based distance between the bounding box and the presentation window.

8. The method of claim 4, wherein the step of determining whether characteristics of the surgical sponge satisfy the acceptance criteria comprises:
determining, with the one or more processors, an area within the bounding box;
comparing, with the one or more processors, the determined area of the bounding box against an acceptable range of bounding box area, wherein the acceptable range of the bounding box area is based on the identified sponge type of the surgical sponge;
identifying, with the one or more processors, the presented surgical sponge to be too close or too far from the optical sensor if the determined area of the bounding box is outside the acceptable range of the bounding box area; and
preventing the optical sensor from capturing of a QBL image of the presented surgical sponge that is too close or too far.

9. The method of claim 1, wherein the step of determining whether characteristics of the surgical sponge satisfy the acceptance criteria comprises:
generating, with the one or more processors, a segmentation mask of the presented surgical sponge;
determining, with the one or more processors, a number of pixels in the segmentation mask;
comparing, with the one or more processors, the determined number of pixels of the segmentation mask against an acceptable range of pixels, wherein the acceptable range of the number of pixels is based on the identified sponge type of the surgical sponge;
identifying, with the one or more processors, an invalid mask condition if the determined number of pixels of the segmentation mask is less than a lower limit of the acceptable range of the number of pixels; and
preventing the optical sensor from capturing of a QBL image of the presented surgical sponge with an invalid mask condition.

10. The method of claim 1, wherein the system includes a depth sensor, and wherein step of determining whether characteristics of the surgical sponge satisfy the acceptance criteria comprises:
generating, with the one or more processors, a segmentation mask of the presented surgical sponge;
determining, with the one or more processors, a real-world sponge area of the segmentation mask based on depth data from the depth sensor;
comparing, with the one or more processors, the determined real-world sponge area of the segmentation mask against an acceptable range of sponge area, wherein the acceptable range of sponge area is based on the identified sponge type of the surgical sponge;
identifying, with the one or more processors, the presented surgical sponge as a wrong sponge if the determined real-world sponge area of the segmentation mask is outside the acceptance range of the sponge area; and
preventing, with the one or more processors, the optical sensor from capturing of a QBL image of the wrong sponge.

11. The method of claim 10, further comprising rejecting, with the one or more processors, pixels from the segmentation mask if estimated differences in depth values between respective points and plane fitted to the depth map is outside of a threshold.

12. (canceled)

13. The method of claim 1, wherein the step of determining whether characteristics of the surgical sponge satisfy the acceptance criteria comprises:
generating, with the one or more processors, a segmentation mask of the presented surgical sponge;
determining, with the one or more processors, a centroid the segmentation mask;
determining, with the one or more processors, an average motion magnitude of the centroid within a predetermined number of image frames of the image feed;
comparing, with the one or more processors, the determined average motion magnitude of the centroid against a centroid change threshold;
identifying, with the one or more processors, the presented surgical sponge as a moving sponge if the determined average motion magnitude of the centroid is greater than the centroid change threshold; and
preventing the optical sensor from capturing of a QBL image of the moving sponge.

14. The method of claim 1, wherein the step of determining whether characteristics of the surgical sponge satisfy the acceptance criteria comprises:
detecting, with the one or more processors, a torso of a user in the field of view of the optical sensor;
identifying, with the one or more processors, a body centerline of the torso of the user and a vertical centerline of the presented surgical sponge;
determining, with the one or more processors, a horizontal shift of the vertical centerline relative to body centerline;
comparing, with the one or more processors, the horizontal shift against a horizontal shift threshold; and
displaying, on the user interface, guidance to instruct to the user to move the presented surgical sponge if the horizontal shift exceeds the horizontal shift threshold.

15. The method of claim 14, wherein the step of determining whether characteristics of the surgical sponge satisfy the acceptance criteria further comprises:
identifying, with the one or more processors, an additional centerline of the user and a horizontal centerline of the presented surgical sponge;
determining, with the one or more processors, a vertical shift of the horizontal centerline relative to the additional centerline;
comparing, with the one or more processors, the vertical shift against a vertical shift threshold; and
displaying, on the user interface, guidance to instruct to the user to move the presented surgical sponge if the vertical shift exceeds the vertical shift threshold.

16. The method of claim 1, further comprising:
receiving, the one or more processors, a localization label from a localization neural network and a segmentation label from a segmentation neural network;
generating, the one or more processors, an aggregated sponge label;
executing, the one or more processors, a sliding window protocol to in which a most frequent aggregated sponge label is identified as a dominant label; and
determining, the one or more processors, that the QBL image is ready for capture if the dominant label indicates the acceptance criteria has been satisfied for a preset number of latest consecutive image frames of the image feed.

17. A method of assessing surgical sponges bloodied in a surgical procedure with a sponge management system including a data reader, one or more processors, a user interface, a depth sensor, and an optical sensor, wherein each of the surgical sponges includes a tag storing a unique identifier, the method comprising:
detecting, with the data reader, the tag of the surgical sponge, wherein the unique identifier stored on the tag is received by the one or more processors;
identifying, with the one or more processors, the surgical sponge as counted out from the surgical procedure;
identifying, with the one or more processors, a sponge type of the surgical sponge from a database of predetermined sponge types based on the unique identifier;
capturing, with the optical sensor, a QBL image of the surgical sponge;
estimating, with the one or more processors, a volume of the blood or a blood component on the surgical sponge based on color component values of the QBL image and the identified sponge type; and
displaying, on the user interface, the volume of the blood or the blood component.

18. The method of claim 17, wherein a hemoglobin estimation algorithm is trained for each of the predetermined sponge types, wherein the step of estimating the volume of the blood component further comprises applying, with the one or more processors, a selected one of the hemoglobin estimation algorithms that corresponds to the identified sponge type.

19. A method of assessing surgical sponges bloodied in a surgical procedure with a sponge management system including a data reader, one or more processors, a user interface, an optical sensor, wherein each of the surgical sponges includes a tag storing a unique identifier, the method comprising:
detecting, with the data reader, the tag of the surgical sponge, wherein the unique identifier stored on the tag is received by the one or more processor;
identifying, with the one or more processors, a sponge type of the surgical sponge from a database of predetermined types of surgical sponges based on the unique identifier;
displaying, on the user interface, a presentation window overlying an image feed being captured by the optical sensor, wherein dimensions of the presentation window are based on the identified sponge type of the surgical sponge;
capturing, with the optical sensor, a QBL image of the surgical sponge once the surgical sponge is presented within the presentation window;
estimating, with the one or more processors, a volume of the blood or a blood component on the surgical sponge based on the QBL image; and
displaying, on the user interface, the volume of the blood or the blood component.

20-27. (canceled)

28. The method of claim 19, further comprising providing, on the user interface, instructions to manipulate the presented surgical sponge according to a folding protocol based on the identified sponge type, wherein the dimensions of the presentation window are further based on dimensions of the surgical sponge following the folding protocol.

29. The method of claim 1, further comprising automatically activating the optical sensor based on the detection of the tag.
